(19) United States
(12) Patent Application Publication
(10) Pub. No.: US 2025/0064179 A1
(43) Pub. Date: Feb. 27, 2025

(54) WATCH STRAP, WATCH ACCESSORY, AND PROTECTIVE SHELL

(71) Applicant: Ningbo Kangluo Intelligent Technology Co.Ltd., Ningbo (CN)

(72) Inventor: HAO ZHENG, Ningbo City (CN)

(21) Appl. No.: 18/422,246

(22) Filed: Jan. 25, 2024

(30) Foreign Application Priority Data
Aug. 23, 2023 (CN) 202311070804.4
Jan. 4, 2024 (CN) 202410016666.X

(51) Int. Cl. A44C 5/00; A61H 39/00; A61H 39/04

(52) U.S. Cl. CPC A44C 5/0023; A61H 39/002; A61H 39/04; A61H 9/0078; A61H 2201/1409; A61H 2201/5071; A61H 2205/06

(57) ABSTRACT

The present disclosure provides a watch strap, a watch accessory and a protective shell. The watch strap includes a watch strap main body, a circuit board and an electrode assembly. Two ends of the watch strap main body are detachably connected to a watch head. The circuit board arranged on the watch strap main body. The electrode assembly arranged on the watch strap main body and is electrically connected to the circuit board for being in contact with a human skin surface and providing a micro current. The problem that it is inconvenient to wear the watch due to the dependence of an existing anti-carsickness bracelet on a main unit to achieve electrical stimulation on acupuncture points is solved.

### CROSS-REFERENCE TO RELATED APPLICATIONS

The application claims priority of Chinese patent application CN202410016666X, filed on Jan. 4, 2024, and also claims priority of Chinese patent application CN2023110708044, filed on Aug. 23, 2023, which are incorporated herein by references in their entireties.

### TECHNICAL FIELD

The present disclosure relates to the technical field of watch parts, and in particular, to a watch strap, a watch accessory, and a protective shell.

### BACKGROUND

At present, the existing anti-carsickness bracelets generally include a main unit and strap bodies arranged on two sides of the main unit. The main unit is worn on the wrist of a user through the strap bodies, and the main unit is provided with an electrical stimulation mechanism to electrically stimulate the Neiguan acupuncture point on the wrist. An electrical stimulation current signal is transmitted to a nerve through another nerve, and a signal of nausea and vomiting is suppressed through the nerves. However, the current method relies on the main unit to achieve the electrical stimulation on acupuncture points. After the anti-carsickness bracelet is worn, it is inconvenient to wear a watch. This affects the use experience of the user.

### SUMMARY

The present disclosure aims to provide a watch strap, a watch accessory and a protective shell. The problem that it is inconvenient to wear the watch due to the dependence of an existing anti-carsickness bracelet on a main unit to achieve electrical stimulation on acupuncture points is solved.

In order to solve the technical problem, the technical scheme provided by the present disclosure is as follows.

In the first aspect, the present disclosure provides a watch strap, including: a watch strap main body, wherein two ends of the watch strap main body are detachably connected to a watch head; a circuit board, arranged on the watch strap main body; and an electrode assembly, arranged on the watch strap main body and is electrically connected to the circuit board for being in contact with a human skin surface and providing a micro current.

Further, the circuit board is a flexible circuit board, and the flexible circuit board is arranged inside the watch strap main body.

Further, the watch strap further includes at least one driving device arranged on the watch strap main body and electrically connected to the circuit board, and the driving device is configured to drive the electrode assembly to abut against the human skin surface.

Further, the electrode assembly is arranged on the driving device, and the driving device is arranged on the watch strap main body.

Further, the driving device includes a first airbag arranged on the watch strap main body and an air pump configured to inflate and deflate the first airbag; the first airbag drives the electrode assembly to abut against the skin surface during inflation; and the electrode assembly is arranged on the first airbag or the watch strap main body.

Further, the watch strap further includes: a battery and a wireless charging coil which are electrically connected to the circuit board and arranged inside the watch strap main body, wherein the battery is configured to supply power to the circuit board.

Further, the two ends of the watch strap main body are provided with connectors detachably connected to the watch head.

Further, the watch strap further includes a second airbag and/or protruding part for pressing acupuncture points, wherein the second airbag is a inflatable airbag.

Further, the watch strap further includes a second airbag for pressing acupuncture points, wherein the first airbag and the second airbag are of an integrated structure, and the first airbag is communicated to the second airbag; and the first airbag and/or the second airbag are provided with inflation and deflation ports.

Further, the watch strap further includes a micro control unit (MCU) arranged on the circuit board and electrically connected to the driving device and the electrode assembly respectively, a pressure sensor electrically connected to the MCU, and an air pressure sensor electrically connected to the MCU; the pressure sensor is configured to: obtain a contact pressure value between the electrode assembly and the human skin surface to transmit the contact pressure value to the MCU; the MCU generates driving data for the air pump on the basis of the contact pressure value; the air pump inflates and/or deflates the first airbag according to the driving data, so that the electrode assembly abuts against or is released from the human skin surface; the air pressure sensor is configured to sense an air pressure value generated by the driving device and transmit the air pressure value to the MCU; and the MCU is configured to determine an electrical stimulation signal applied to the human skin surface for the driving data and the electrode assembly according to the air pressure value and the current contact pressure value of the electrode assembly.

Further, the MCU is further configured to: generate, in a case that it is determined that the contact pressure value between the electrode assembly and the human skin surface is less than or greater than a preset pressure value, a reminder signal for reminding a user to tighten the watch strap.

Further, the watch strap further includes a display screen electrically connected to the MCU, wherein the display screen is configured to display a display result of a target task in response to a display instruction performed by the user wearing the watch strap on the target task, and the display result at least includes one or more of the following information: electric stimulation intensity, an electrode ejection force, countdown protection information to avoid long-term use by the user, a breathing pattern, and user health data; the display screen is a touch display screen; the touch display screen is configured to display a control result of the target task in response to a touch control instruction performed by the user wearing the watch strap on the target task; and the control result includes changing one or more of the electric stimulation massage intensity, a pressure, countdown time, and time under touch control.

Further, the watch strap further includes a health data acquisition assembly electrically connected to an MCU, wherein the health data acquisition assembly is configured to acquire in real time or periodically health data of the user wearing the watch strap; the health data at least includes one or more of the following data: a body temperature, a blood pressure, blood oxygen, and a heartbeat; and the MCU provides health analysis and health reports according to the health data.

Further, the MCU is configured to obtain driving data for the driving device; in a case that the driving device causes the watch strap to compress the human skin according to the driving data, the MCU applies an electrical stimulation signal to the electrode assembly to apply electrical stimulation to the human skin; and wherein the MCU is also configured to apply the electrical stimulation to the electrode assembly in a case of determining that a preset electrical stimulation condition is satisfied.

Further, the watch strap further includes a switch component electrically connected to the MCU, wherein the switch component is configured to obtain a switch signal triggered by the user and transmit the switch signal to the MCU; and the MCU is configured to determine an open/closed state of the watch strap on the basis of a continuous state of the switch signal, and/or to determine an electrical stimulation mode and intensity of the electrode assembly.

Further, the watch strap further includes a rotary encoder electrically connected to the MCU, wherein the rotary encoder is configured to: determine a degree of the electrical stimulation for the electrode assembly on the basis of a variation indicated by a rotation adjustment operation in response to the rotation adjustment operation, so that the MCU applies the electrical stimulation that is matched with the degree of the electrical stimulation to the electrode assembly; or, determine a force for ejecting the electrode assembly, so that the MCU applies a matching pressure to the electrode assembly; or, determine time or countdown time for ejecting the electrode assembly, so that the MCU takes matching pressing time to the electrode assembly.

In another aspect, the present disclosure also provides a watch accessory, including: a protective shell, wherein a mounting slot configured to place the watch head is formed in the protective shell; a strap body, wherein two ends of the strap body are connected to the protective shell; a circuit board, arranged on the strap body or the protective shell; and an electrode assembly, arranged on the strap body or the protective shell and is electrically connected to the circuit board for being in contact with a human skin surface and providing a micro current.

Further, the watch accessory further includes at least one driving device arranged on the strap body or the protective shell, wherein the driving device is configured to drive the electrode assembly to abut against the human skin surface.

Further, the electrode assembly is arranged on the driving device, and the driving device is arranged on the strap body or the protective shell.

In another aspect, the present disclosure also provides a protective shell, including: a shell body, wherein the shell body is provided with a mounting slot for placing a watch head; a circuit board, arranged on the shell body; and an electrode assembly, arranged on the shell body and is electrically connected to the circuit board for being in contact with a human skin surface and providing a micro current.

The present disclosure has the following beneficial effects. Compared with the prior art, In this present disclosure, since the circuit board and the electrode assembly are configured on the watch accessory detachably connected to a watch, when a user uses the watch accessory in this embodiment in conjunction with the watch head and wears it on the wrist, the electrode assembly is driven by the circuit board to provide electrical stimulation to the Neiguan acupuncture point on the wrist, and can also have the function of the watch, and a user does not need to wear the watch. Furthermore, the problem that it is inconvenient to wear the watch due to the dependence of an existing anti-carsickness bracelet on a main unit to achieve electrical stimulation on acupuncture points is solved.

### BRIEF DESCRIPTION OF THE DRAWINGS

In order to explain the technical solutions of the embodiments of the present disclosure more clearly, the following will briefly introduce the accompanying drawings used in the embodiments. Apparently, the drawings in the following description are only some embodiments of the present disclosure. Those of ordinary skill in the art can obtain other drawings based on these drawings without creative work.

FIG. 1 is a schematic diagram of a watch strap main body according to the present disclosure;

FIG. 2 is an exploded view of the present disclosure;

FIG. 3 is a schematic diagram of another embodiment according to the present disclosure;

FIG. 4 is a schematic diagram of connection with a watch head according to the present disclosure;

FIG. 5 is an exploded diagram of a driving device, an electrode assembly, and a protective shell according to the present disclosure;

FIG. 6 is a schematic diagram of assembling a driving device and an electrode assembly on a protective shell according to the present disclosure;

FIG. 7 is a cross-sectional view of assembling a driving device and an electrode assembly on a protective shell according to the present disclosure;

FIG. 8 is a structural diagram of a driving device in an embodiment according to the present disclosure;

FIG. 9 is a structural diagram of a driving device in an embodiment according to the present disclosure;

FIG. 10 is a structural diagram of a driving device in an embodiment according to the present disclosure;

FIG. 11 is a structural diagram of a driving device in an embodiment according to the present disclosure;

FIG. 12 is a structural diagram of a driving device in an embodiment according to the present disclosure;

FIG. 13 is a structural diagram of a driving device in an embodiment according to the present disclosure; and

FIG. 14 is a structural diagram of a driving device in an embodiment according to the present disclosure.

### DETAILED DESCRIPTION OF THE EMBODIMENTS

The accompanying drawings in the embodiment of the present disclosure are combined, The technical scheme in the embodiment of the present disclosure is clearly and completely described. Obviously, the described embodiment is only a part of the embodiment of the present disclosure, but not all embodiments are based on the embodiment of the present disclosure, and all other embodiments obtained by ordinary technicians in the field on the premise of not doing creative work belong to the protection range of the present disclosure.

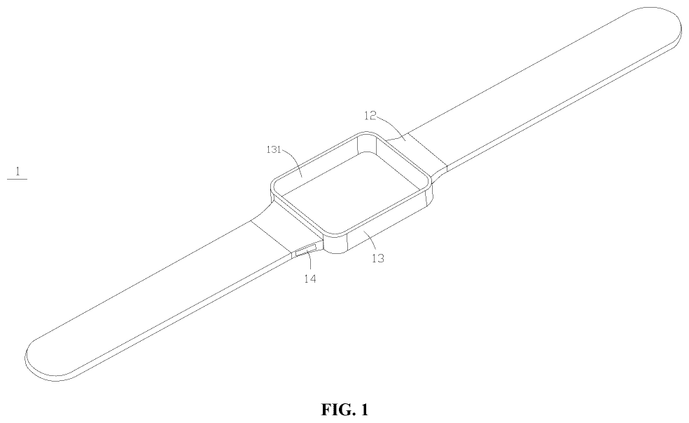

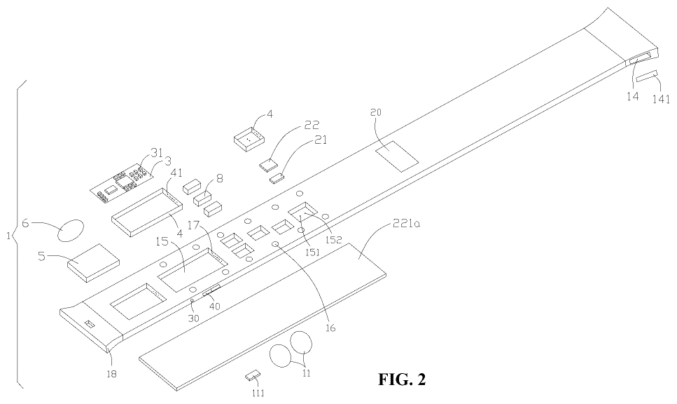

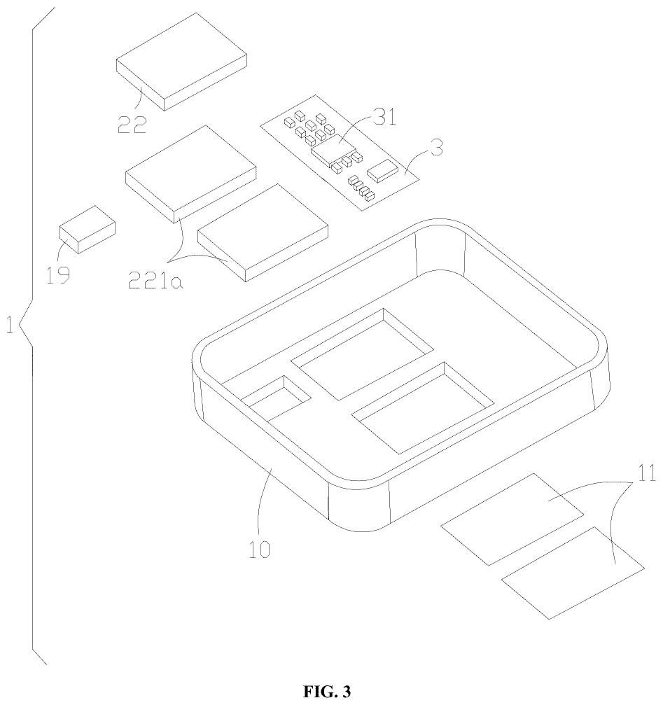

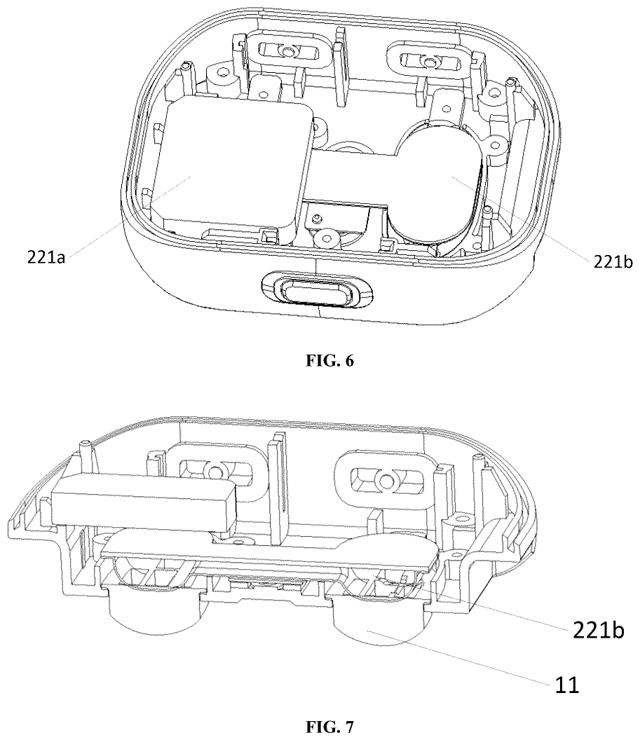

Referring to FIG. 1 to FIG. 14, the embodiments of the present disclosure provide a watch accessory.

Referring to FIG. 1 to FIG. 4, the watch accessory includes: a watch strap main body 1, detachably connected to a watch head 00; a circuit board 3, arranged on the watch strap main body 1; and an electrode assembly 11, arranged on the watch strap main body 1 and is electrically connected to the circuit board 3 for being in contact with a human skin surface and providing a micro current.

In this embodiment, since the circuit board 3 and the electrode assembly 11 are configured on the watch accessory detachably connected to a watch, when a user uses the watch accessory in this embodiment in conjunction with the watch head 00 and wears it on the wrist, the electrode assembly 11 is driven by the circuit board 3 to provide electrical stimulation to the Neiguan acupuncture point on the wrist, and can also have the function of the watch, and a user does not need to wear the watch. Furthermore, the problem that it is inconvenient to wear the watch due to the dependence of an existing anti-carsickness bracelet on a main unit to achieve electrical stimulation on acupuncture points is solved.

In an embodiment, the circuit board 3 is a flexible circuit board 3, and the flexible circuit board 3 is arranged inside the watch strap main body 1. The flexible circuit board 3 is flexible and can be bent to adapt to the watch accessory for use. When the watch accessory is fixed on the wrist of the user, the circuit board 3 does not affect the use of the watch accessory and allows the watch accessory to abut against the wrist.

In an embodiment, the watch accessory further includes at least one driving device 2 arranged on the watch strap main body 1 and electrically connected to the circuit board 3, and the driving device 2 is configured to drive, according to a control signal output by the circuit board 3, the electrode assembly 11 to abut against a human skin surface. When the user uses the electrical stimulation, the electrode assembly 11 can be driven by the driving device 2 to abut against the human skin surface, so as to effectively improve the electrical stimulation effect of the electrode assembly 11.

Specifically, in the above embodiment, the electrode assembly 11 is arranged on the driving device 2, and the driving device 2 is arranged on the watch strap main body 1, so that when driven by the driving device 2, the electrode assembly 11 can abut against the human skin surface. Moreover, this can solve the discomfort caused by the existing method of tightening a strap body 12 on the wrist to cause the electrode assembly 11 to abut against the human skin surface, and improve the comfort of wearing of the user. Specifically, in the above embodiments, the electrode assembly 11 is not arranged on the driving device 2, that is, the electrode assembly 11 and the driving device 2 are both arranged on the watch strap main body 1. Specifically, the electrode assembly 11 and the driving device 2 can be arranged at approximately opposite positions, so that when driven by the driving device 2, the electrode assembly 11 can also abut against the human skin surface.

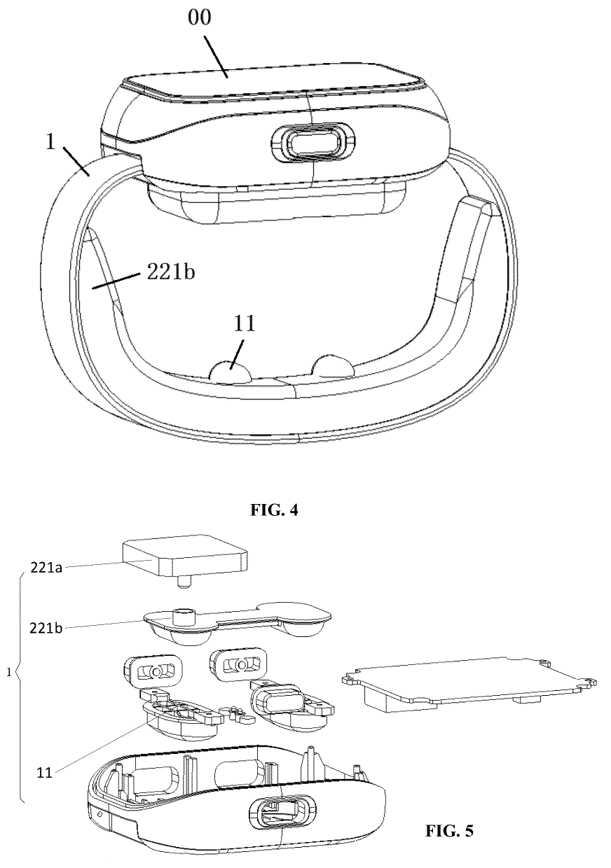

In an embodiment, referring to FIG. 4 to FIG. 7, the driving device 2 includes a first airbag 221b arranged on the watch strap main body 1 and an air pump 221a configured to inflate and deflate the first airbag 221b; the first airbag 221b drives the electrode assembly 11 to abut against the skin surface during inflation; and the electrode assembly 11 is arranged on the first airbag 221b or the watch strap main body 1. The first airbag 221b itself has elasticity, which can effectively improve the comfort of wearing of the user.

Specifically, the first airbag 221b can be long-strip-shaped and is arranged in a lengthwise direction of the watch strap main body 1. Or, a surface of the first airbag 221b may only be slightly larger than a surface of the electrode assembly 11.

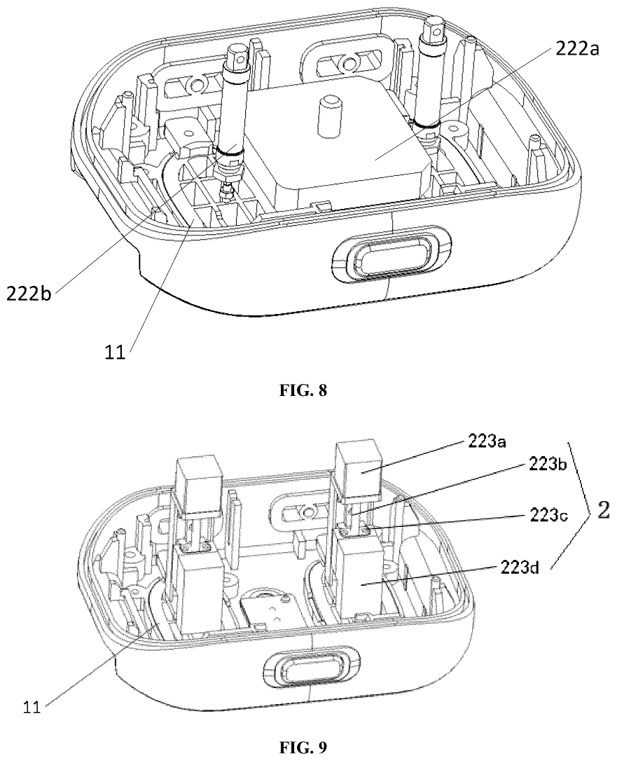

In other embodiments, referring to FIG. 8, the driving device 2 may further include an air pump 221a and an air cylinder 222b connected to the air pump 221a through an air pipe. The air cylinder 222b drives the electrode assembly 11 to abut against the skin surface when the air pump 221a blows air to the air cylinder. The air pump 221a is electrically connected to the circuit board 3, and the effect that the electrode assembly 11 abuts against the skin surface can also be achieved. Specifically, a micro air cylinder can be used here.

In other embodiments, referring to FIG. 9, the driving device 2 includes a motor 223a, a screw rod 223b connected to an output shaft of the motor 223a, a screw nut 223c used in conjunction with the screw rod 223b, and a sliding block 223d arranged on the screw nut 223c. The motor 223a is electrically connected to the circuit board 3, and the sliding block 223d drives the electrode assembly 11 to abut against the skin surface when the screw rod 223b rotates. This can also achieve the effect that the electrode assembly 11 abuts against the skin surface.

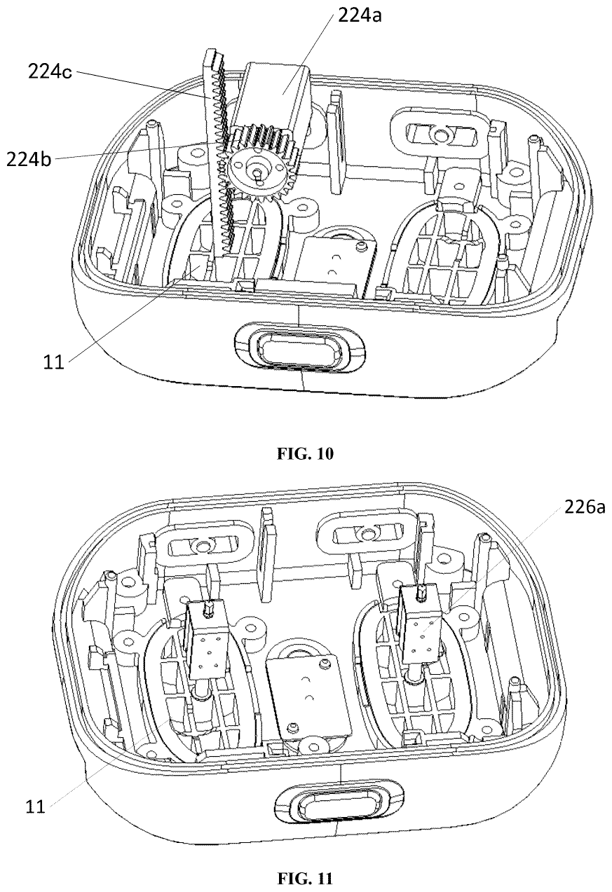

In other embodiments, referring to FIG. 10, the driving device 222 includes a motor 224a, a gear 224b connected to the motor 224a, and a rack 224c engaged with the gear 224b. The motor 223a is electrically connected to the circuit board 3. The rack 224c drives the electrode assembly 11 to abut against the skin surface when the gear 224b rotates. This can also achieve the effect that the electrode assembly 11 abuts against the skin surface.

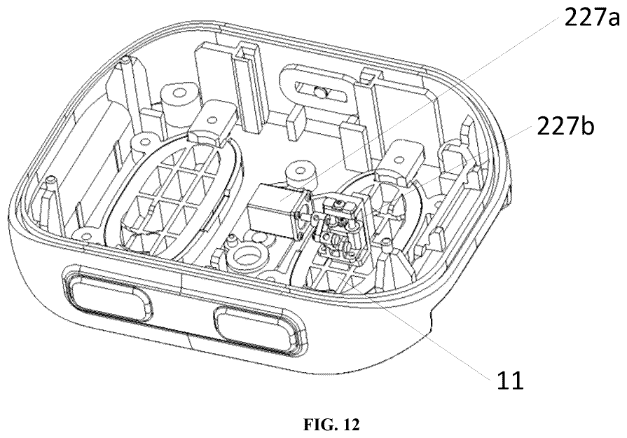

In other embodiments, referring to FIG. 11, the driving device 222 includes a coil 226a and a magnet. When the coil 226a is energized, the magnet moves to drive the electrode assembly 11 to abut against the skin surface. This can also achieve the effect that the electrode assembly 11 abuts against the skin surface. Specifically, the electrode assembly 11 can be a conductive magnet with a polarity that repels the coil 226a. The coil 226a is electrically connected to the circuit board 3.

In other embodiments, referring to FIG. 12, the driving device 222 includes a motor 227a and a crank 227b electrically connected to the motor 227a. When the crank 227b is driven by the motor 227a, the electrode assembly 11 is driven to abut against the skin surface.

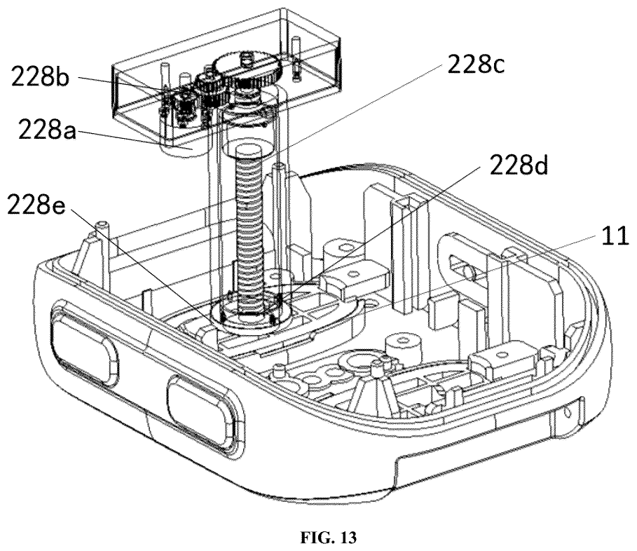

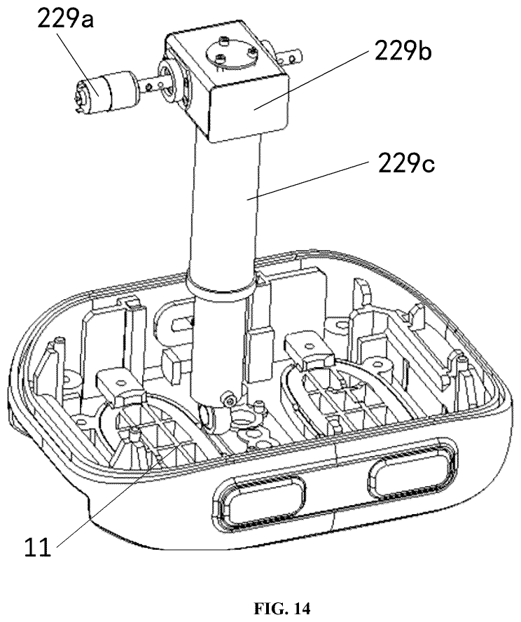

In other embodiments, referring to FIG. 13, the driving device 222 includes a motor 228a, a gear 228b connected to the motor 228a and having a screw rod 228c, a screw nut 228d movably arranged on the screw rod 228c, and a sliding block 228e arranged on the screw nut 228d. The gear 228b drives the screw rod 228c to rotate when driven by the motor 228a. The screw nut 228d drives the sliding block 228e to move when the screw rod 228c rotates, so as to drive the electrode assembly 11 to abut against the skin surface. The motor 228a is electrically connected to the circuit board 3.

In other embodiments, referring to FIG. 14, the driving device 222 includes a motor 229a, a worm 229b connected to the motor 229a, and a screw rod 229c connected to the worm 229b. When the worm 229b rotates, the screw rod 229c drives the electrode assembly 11 to abut against the skin surface through a nut on the screw rod, and the motor 229 is electrically connected to the circuit board 3.

In the above embodiment, the driving device 2 can be arranged on a surface of the watch strap main body 1. Or, a slot is formed in the watch strap main body 1 to assemble the driving device 2. Or, a watch body is provided with a shell, and the driving device 2 is arranged inside the shell.

Referring to FIG. 1 to FIG. 4, in an embodiment, the watch accessory further includes: a battery 5 and a wireless charging coil 6 which are electrically connected to the circuit board 3 and arranged inside the watch strap main body 1, wherein the battery 5 is configured to supply power to the circuit board 3. The wireless charging coil 6 is used in conjunction with an existing wireless charger for a smart watch on the market, input power charges the battery 5 through the circuit board 3. Of course, in other embodiments, the battery 5 may also be a non-rechargeable button battery 5. Or, in the embodiment with the battery 5, the watch accessory also includes a charging contact electrically connected to the circuit board 3. The charging contact can also be configured to charge the battery 5 when being connected to an external adapter.

In an embodiment, connectors 18 detachably connected to the watch head 00 are arranged at two ends of the watch strap main body 1, so as to achieve effects off connecting the watch strap main body 1 to the watch head 00 and replacing the watch head 00. Specifically, the connectors 18 can be the same as a connection structure between a watch accessory on an Apple watch and a watch head 00, or as connection structures between other watch accessories and watch heads 00 on the market. The structures of the connectors 18 and the detachable structure between the watch accessory and the watch head 00 will not be elaborated here.

In an embodiment, the watch strap main body 1 includes a protective shell 13 and a strap body 12, two ends of which are connected to the protective shell 13. The protective shell 13 is provided with a mounting slot 131 configured to place the watch head 00. The circuit board 3 is arranged on the strap body 12 or the protective shell 13. The electrode assembly 11 is arranged on the strap body 12 or the protective shell 13 and is electrically connected to the circuit board 3 for being in contact with the human skin surface and providing a micro current. The watch head 00 is mounted in the mounting slot 131, so that the watch accessory of this embodiment can be detachably connected to the watch head 00.

Specifically, the driving device 2 is arranged on the protective shell 13, and the electrode assembly 11 is arranged on the strap body 12. Or, the driving device 2 is arranged on the strap body 12, and the electrode assembly 11 is arranged on the protective shell 13. Or, the electrode assembly 11 is arranged on the driving device 2. The driving device 2 is arranged on the strap body 12 or the protective shell 13, to drive the electrode assembly 11 to abut against the skin by the driving device 2.

In an embodiment, the watch strap main body 1 includes a shell body 10 detachably connected to the watch strap, so that this embodiment can be worn on the wrist through the watch strap. The mounting slot 131 configured to place the watch head 00 is formed in the shell body 10. The watch head 00 is mounted in the mounting slot 131, so that the watch accessory of this embodiment is detachably connected to the watch head 00. Specifically, the circuit board 3 is arranged on the shell body 10, and the electrode assembly 11 is arranged on the shell body 10 and is electrically connected to the circuit board 3 for being in contact with the human skin surface and providing the micro current.

In an embodiment, the watch accessory further includes a second airbag 19 configured to press an acupuncture point. That is, after the electrode assembly 11 is driven through the first airbag 221b to achieve electrical stimulation, the user can also press the electrically stimulated acupuncture point through the second airbag 19, which further stimulates the acupuncture point to achieve a better acupuncture point stimulation effect.

The second airbag 19 may not be connected to the air pump 221a. The user can stimulate the acupuncture point by pressing the second airbag 19 as needed. Of course, the second airbag 19 may also be connected to the air pump 221a to achieve inflation under the action of the air pump 221a, so as to achieve an effect of stimulating the acupuncture point. In addition, the second airbag 19 can also have the same mode as that of the first airbag 221b. That is, the second airbag achieves inflation and deflation along with the first airbag 221b to achieve synchronous actions with the first airbag 221b. Or, the second airbag and the first airbag 221b can achieve inflation and deflation separately, so as to provide more usage modes for the user and improve the use experience.

In an embodiment, there are a plurality of first airbags 221a. A quantity of the electrode assemblies 11 is equal to the quantity of the first airbags 221a. One electrode assembly 11 corresponds to one first airbag 221b, and a size of the first airbag 221b can be much larger than that of the electrode assembly 11. For example, the first airbags 221a can extend in a direction of the watch accessory. That is, the first airbags occupy an inner side surface of the entire watch accessory. Of course, in other embodiments, there can also be one the first airbag 221b. At this time, a length and width of the electrode assembly 11 can increase relatively to effectively press the acupuncture point on the wrist. Specifically, there can be two first airbags 221a, which are respectively located on two sides of the second airbag 19.

In an embodiment, the first airbag 221b and the second airbag 19 are both inflatable airbags. That is, the first airbag 221b and the second airbag 19 can press, stimulate, and massage the acupuncture point on the wrist through inflation and deflation.

Specifically, in an embodiment, both the first airbag 221b and the second airbag 19 are provided with inflation and deflation interfaces. In other embodiments, the first airbag 221b and the second airbag 19 can be of an integrated structure, and the first airbag 221b is communicated to the second airbag 19. Therefore, an inflation and deflation interface is arranged on the first airbag 221b and/or the second airbag 19. The inflation and deflation interface is connected to the air pump 221a to achieve inflation and deflation of the first airbag 221b and the second airbag 19.

In an embodiment, the watch accessory may further include at least one protruding part 111 configured to press the acupuncture point. Specifically, the protruding part 111 is a protruding point, a metal sheet, and the like. The protruding part 111 is arranged on the first airbag 221b to achieve massage when the first airbag 221b is inflated. Of course, in other embodiments, the protruding part 111 can also be arranged on the second airbag 19, the watch head 00, or the watch accessory. The position of the protruding part 111 will not be limited here.

In an embodiment, the watch accessory further includes a micro control unit (MCU) 31 arranged on the circuit board 3 and electrically connected to the driving device 2 and the electrode assembly 11 respectively, a protruding part 111 electrically connected to the MCU 31, and an air pressure sensor 21 electrically connected to the MCU. The protruding part 111 is configured to obtain a contact pressure value between the electrode assembly 11 and the human skin surface, and transmit the contact pressure value to the MCU 31. The MCU 31 generates driving data for the air pump 221a on the basis of the contact pressure value. The air pump 221a inflates and/or deflates the first airbag 221b according to the driving data, so as to cause the electrode assembly 11 abut against or be released from the human skin surface. The air pressure sensor 21 is configured to sense an air pressure value generated by the driving device 2 and transmit the air pressure value to the MCU 31. The MCU 31 is configured to determine an electrical stimulation signal applied to the human skin surface for the driving data and the electrode assembly 11 according to the air pressure value and the current contact pressure value of the electrode assembly 11.

Specifically, the air pressure sensor 21 can be arranged inside the first airbag 221b. In this way, the air pressure sensor 21 can detect the air pressure value inside the first airbag 221b in real time. The MCU 31 can determine the driving data suitable for the air pump 221a according to the air pressure value and the contact pressure value measured by the protruding part 111.

In this embodiment, the MCU 31 reads the measured value of the air pressure sensor 21 inside the first airbag 221b, thereby effectively controlling an air pressure inside the first airbag 221b through the air pump 221a. On the one hand, a precise instantaneous air pressure fluctuation curve can be used to simulate a physical point-pressing function of traditional Chinese medicine. The excitation of the median nerve is enhanced through physical point-pressing, and the effectiveness of stimulation can be significantly improved. On the other hand, changes of the air pressure inside the first airbag 221b can be quickly switched, and the hardness of the first airbag 221b and a depth of the top-concave skin can be operated to change, to simulate the function of physically repeated pressing and vibration rubbing. This function can effectively activate the median nerve deep in the wrist in combination with the electrical stimulation, achieving an overlay effect of auxiliary massage, acupuncture and moxibustion, or electric moxibustion in traditional Chinese medicine diagnosis and treatment, and further improving the practicability.

In an embodiment, the MCU 31 is further configured to: generate, in a case that it is determined that the contact pressure value between the electrode assembly 11 and the human skin surface is less than or greater than a preset pressure value, a reminder signal for reminding the user to tighten the watch accessory.

In an embodiment, the watch accessory further includes a display screen 20 electrically connected to the MCU 31, wherein the display screen 20 is configured to display a display result of a target task in response to a display instruction performed by the user wearing the watch accessory on the target task, and the display result at least include one or more of the following information: electric stimulation intensity, an electrode ejection force, countdown protection information to avoid long-term use by the user, a breathing pattern, and user health data, so as to facilitate use.

Specifically, the display screen 20 is a touch display screen 20. The display screen 20 is configured to display a control result of the target task in response to a touch control instruction performed by the wearing the watch accessory on the target task. The control result includes changing one or more of the electric stimulation massage intensity, a pressure, countdown time, and time under touch control, so as to facilitate operation and use.

In an embodiment, the watch accessory also includes a health data acquisition assembly 8 electrically connected to the MCU 31. The health data acquisition assembly 8 is configured to acquire in real time or periodically health data of the user wearing the watch accessory; the health data at least includes one or more of the following data: a body temperature, a blood pressure, blood oxygen, and a heartbeat; and the MCU 31 provides health analysis and health reports according to the health data, so as to improve the practicability of the watch accessory. Specifically, the health data acquisition assembly 8 can include a heart rate sensor, a motion sensor, and the like.

In the above implementation, the circuit board 3 can be integrated with the watch strap main body 1 by injection molding. Or, the watch strap main body 1 is provided with grooves 15 at positions corresponding to the circuit board 3, the air pump 221a, the health data acquisition assembly 8, the battery 5, and the wireless charging coil 6. The circuit board 3, the air pump 221a, the health data acquisition assembly 8, the battery 5, and the wireless charging coil 6 are placed in their respective suitable grooves 15 and then are glued, so as to fix the circuit board 3, the air pump 221a, the health data acquisition assembly 8, the battery 5, and the wireless charging coil 6 on the watch strap main body 1. In another embodiment, the watch accessory further includes housings 4 configured to respectively mount the circuit board 3, the air pump 221a, the health data acquisition assembly 8, the battery 5, and the wireless charging coil 6. The housings 4 can be arranged on an outer side of the watch strap main body 1, or the housings 4 can be arranged in the grooves 15 and are in interference fit with the grooves 15 to be fixed. Of course, in other embodiments, the housings can also be clamped in the grooves 15 to be fixed.

In practical applications, each housing 4 is provided with a routing hole 41 that is internally communicated and configured for routing. The watch strap main body 1 is provided with a plurality of routing channels 17. The plurality of routing channels 17 respectively route wires for electrical connection between the circuit board 3, the air pump 221a, the health data acquisition assembly 8, the battery 5, and the wireless charging coil 6, so that the circuit board 3 can be electrically connected to the air pump 221a, the health data acquisition assembly 8, the battery 5, and the wireless charging coil 6 through the wire routing. Further, an air outlet hole and an air inlet hole which are connected to the air pump 221a through the air pipe are further formed in the housing 4 configured to mount the air pump 221a, so that it is convenient to connect the air pump 221a to the airbags.

In an embodiment, the watch strap main body 1 is provided with a receiving slot 14 configured to place medicines, and a cover body 141 is arranged at a notch of the receiving slot 14 to place some small-sized pills for convenient use by the use when the use goes out. Specifically, the receiving slot 14 can be arranged on the strap body 12 or on the protective shell 13.

In an embodiment, the watch accessory further includes a switch component 40 electrically connected to the MCU 31, and the switch component 40 is configured to obtain a switch signal triggered by the user and transmit the switch signal to the MCU 31. The MCU 31 is configured to determine an open/closed state of the watch accessory on the basis of a continuous state of the switch signal, and/or to determine an electrical stimulation mode and intensity of the electrode assembly 11.

Specifically, the switch component 40 can include a first switch electrically connected to the MCU 31 to control an on/off state of a bracelet or to control switching of the electrical stimulation mode of the electrode assembly 11. The switch component 40 can further include a second switch electrically connected to the MCU 31 to control the electrical stimulation intensity of the electrode assembly 11 and the like.

The first switch, the second switch, or another switch can be arranged on a surface or a side edge of the watch strap main body 1. The first switch and the second switch can be physical button switches, virtual touch switches, or the like, and specific limitations are not made here.

In an embodiment, the watch accessory further includes a rotary encoder 30 electrically connected to the MCU 31. The rotary encoder 30 is configured to: determine a degree of the electrical stimulation for the electrode assembly 11 on the basis of a variation indicated by a rotation adjustment operation in response to the rotation adjustment operation, so that the MCU 31 applies the electrical stimulation that is matched with the degree of the electrical stimulation to the electrode assembly 11; or, determine a force for ejecting the electrode assembly 11, so that the MCU 31 applies a matching pressure to the electrode assembly 11; or, determine time or countdown time for ejecting the electrode assembly 11, so that the MCU 31 takes matching pressing time to the electrode assembly 11. Based on the rotary encoder 30, the corresponding rotation control operation can be achieved to further improve the practicality in different application scenarios.

It should be noted that all directional indications (such as up, down, left, right, front, back . . . ) in the embodiments of the present disclosure are only used to explain a relative positional relationship between components, motion situations, etc. at a certain specific attitude (as shown in the figures). If the specific attitude changes, the directional indication also correspondingly changes.

In addition, the descriptions of “first”, “second”, etc. in the present disclosure are only used for descriptive purposes, and cannot be understood as indicating or implying its relative importance or implicitly indicating the number of technical features indicated. Therefore, features defined by “first” and “second” can explicitly instruct or impliedly include at least one feature. In addition, “and/or” in the entire text includes three solutions. A and/or B is taken as an example, including technical solution A, technical solution B, and technical solutions that both A and B satisfy. In addition, the technical solutions between the various embodiments can be combined with each other, but it needs be based on what can be achieved by those of ordinary skill in the art. When the combination of the technical solutions is contradictory or cannot be achieved, it should be considered that such a combination of the technical solutions does not exist, and is not within the scope of protection claimed by the present disclosure.

The above descriptions are only preferred embodiments of the present disclosure, and are not intended to limit the patent scope of the present disclosure. Any equivalent structural transformation made by using the content of the specification and the drawings of the present disclosure under the invention idea of the present disclosure, directly or indirectly applied to other related technical fields, shall all be included in the scope of patent protection of the present disclosure.

1. A watch strap, comprising:
a watch strap main body, wherein two ends of the watch strap main body are detachably connected to a watch head;
a circuit board, arranged on the watch strap main body;
an electrode assembly electrically connected to the circuit board for being in contact with a human skin surface and providing a micro current;
at least one driving device arranged on the watch strap main body and electrically connected to the circuit board, the at least one driving device comprising a first airbag arranged on the watch strap main body and an air pump configured to inflate and deflate the first airbag; and
a protruding part for pressing acupuncture points,
wherein the electrode assembly and the protruding part are arranged on the first airbag or on the watch strap main body, and the first airbag, the electrode assembly, and the protruding part are configured in a manner that when the first airbag is inflated, the electrode assembly abuts against the skin surface and achieves electrical stimulation, and the protruding part massages the skin surface.

2. The watch strap according to claim 1, wherein the circuit board is a flexible circuit board, and the flexible circuit board is arranged inside the watch strap main body.

3. (canceled)

4. (canceled)

5. (canceled)

6. The watch strap according to claim 1, further comprising: a battery and a wireless charging coil which are electrically connected to the circuit board and arranged inside the watch strap main body, wherein the battery is configured to supply power to the circuit board.

7. The watch strap according to claim 1, wherein the two ends of the watch strap main body are provided with connectors detachably connected to the watch head.

8. The watch strap according to claim 1, further comprising a second airbag and, wherein the second airbag is an inflatable airbag.

9. The watch strap according to claim 1, further comprising a second airbag for pressing acupuncture points, wherein the first airbag and the second airbag are of an integrated structure, and the first airbag is communicated to the second airbag; and the first airbag and/or the second airbag are provided with inflation and deflation ports.

10. The watch strap according to claim 1, further comprising a micro control unit (MCU) arranged on the circuit board and electrically connected to the at least one driving device and the electrode assembly respectively, a pressure sensor electrically connected to the MCU, and an air pressure sensor electrically connected to the MCU;
the pressure sensor is configured to: obtain a contact pressure value between the electrode assembly and the human skin surface to transmit the contact pressure value to the MCU;
the MCU generates driving data for the air pump on the basis of the contact pressure value;
the air pump inflates and/or deflates the first airbag according to the driving data, so that the electrode assembly abuts against or is released from the human skin surface;
the air pressure sensor is configured to sense an air pressure value generated by the driving device and transmit the air pressure value to the MCU; and
the MCU is configured to determine an electrical stimulation signal applied to the human skin surface for the driving data and the electrode assembly according to the air pressure value and the current contact pressure value of the electrode assembly.

11. The watch strap according to claim 10, wherein the MCU is further configured to:
generate, in a case that it is determined that the contact pressure value between the electrode assembly and the human skin surface is less than or greater than a preset pressure value, a reminder signal for reminding a user to tighten the watch strap.

12. The watch strap according to claim 10, further comprising a display screen electrically connected to the MCU, wherein the display screen is configured to display a display result of a target task in response to a display instruction performed by a user wearing the watch strap on the target task, and the display result at least comprises one or more of the following information: electric stimulation intensity, an electrode ejection force, countdown protection information to avoid long-term use by the user, a breathing pattern, and user health data;
the display screen is a touch display screen; the touch display screen is configured to display a control result of the target task in response to a touch control instruction performed by the user wearing the watch strap on the target task; and the control result comprises changing of the electric stimulation massage intensity, a pressure, countdown time, or time under touch control of the touch display screen, or a combination thereof.

13. The watch strap according to claim 12, further comprising a health data acquisition assembly electrically connected to an MCU, wherein the health data acquisition assembly is configured to acquire in real time or periodically health data of the user wearing the watch strap; the health data at least comprises one or more of the following data: a body temperature, a blood pressure, blood oxygen, and a heartbeat; and the MCU provides health analysis and health reports according to the health data.

14. The watch strap according to claim 10, wherein
the MCU is configured to obtain driving data for the at least one driving device;
in a case that the driving device causes the watch strap to compress the human skin according to the driving data, the MCU applies an electrical stimulation signal to the electrode assembly to apply electrical stimulation to the human skin; and
wherein the MCU is also configured to apply the electrical stimulation to the electrode assembly in a case of determining that a preset electrical stimulation condition is satisfied.

15. The watch strap according to claim 1, further comprising a switch component electrically connected to the MCU, wherein the switch component is configured to obtain a switch signal triggered by a user and transmit the switch signal to the MCU; and the MCU is configured to determine an open/closed state of the watch strap on the basis of a continuous state of the switch signal, and/or to determine an electrical stimulation mode and intensity of the electrode assembly.

16. The watch strap according to claim 15, further comprising a rotary encoder electrically connected to the MCU, wherein the rotary encoder is configured to: determine a degree of the electrical stimulation for the electrode assembly on the basis of a variation indicated by a rotation adjustment operation in response to the rotation adjustment operation, so that the MCU applies the electrical stimulation that is matched with the degree of the electrical stimulation to the electrode assembly; or, determine a force for ejecting the electrode assembly, so that the MCU applies a matching pressure to the electrode assembly; or, determine time or countdown time for ejecting the electrode assembly, so that the MCU takes matching pressing time to the electrode assembly.

17. A watch accessory, comprising:
a protective shell, wherein a mounting slot configured to place the watch head is formed in the protective shell;
a strap body, wherein two ends of the strap body are connected to the protective shell;
a circuit board, arranged on the strap body or the protective shell; and
an electrode assembly, arranged on the strap body or the protective shell and is electrically connected to the circuit board for being in contact with a human skin surface and providing a micro current.

18. The watch accessory according to claim 17, further comprising at least one driving device arranged on the strap body or the protective shell, wherein the driving device is configured to drive the electrode assembly to abut against the human skin surface.

19. The watch accessory according to claim 18, wherein the electrode assembly is arranged on the driving device, and the driving device is arranged on the strap body or the protective shell.

20. A protective shell, comprising:
a shell body, wherein the shell body is provided with a mounting slot for placing a watch head;
a circuit board, arranged on the shell body; and
an electrode assembly, arranged on the shell body and is electrically connected to the circuit board for being in contact with a human skin surface and providing a micro current.

21. A watch strap, comprising:
a watch strap main body, wherein two ends of the watch strap main body are detachably connected to a watch head;
a circuit board, arranged on the watch strap main body;
an electrode assembly electrically connected to the circuit board for being in contact with a human skin surface and providing a micro current;
at least one driving device arranged on the watch strap main body and electrically connected to the circuit board, the at least one driving device comprising a first airbag arranged on the watch strap main body, and an air pump configured to inflate and deflate the first airbag; the electrode assembly being arranged on the first airbag or directly on the watch strap main body, and the first airbag is capable of driving the electrode assembly to abut against the skin surface during inflation; and
a second airbag arranged on the watch strap main body, wherein the first airbag, the second airbag, and the electrode assembly are configured in a manner that after the electrode assembly is driven through the first airbag to achieve electrical stimulation on an acupuncture point, the electrically stimulated acupuncture point are further pressed through the second airbag.
